(19) United States
(12) Patent Application Publication
(10) Pub. No.: US 2012/0015851 A1
(43) Pub. Date: Jan. 19, 2012

(54) METHOD AND DEVICE FOR RAPID, DETECTION OF SPECIFIC REACTIONS USING NON-MAGNETIC AND MAGNETIC NANOPARTICLES

(75) Inventors: Roy Rolando Mondesire, Boulder, CO (US); Antonio Domenico Valdevit, Effort, PA (US)

(73) Assignee: RoMonics, LLC, Boulder, CO (US)

(21) Appl. No.: 13/184,125

(22) Filed: Jul. 15, 2011

Related U.S. Application Data
(60) Provisional application No. 61/364,646, filed on Jul. 15, 2010.

(51) Int. Cl. C12M 1/34; G01N 21/75; C40B 60/12; C12M 1/40

(52) U.S. Cl. 506/39; 435/287.2; 435/288.7; 422/69; 422/82.05

(57) ABSTRACT

A method and apparatus for conducting specific binding assays with two or more particles is claimed. A non-magnetic nanoparticle capable of binding the target of interest is mixed with a magnetic particle capable of binding a species directed toward binding the same target of interest. Following the application of the mixture to a capillary tube or other container that allows the separation with a magnetic field or other force, the resultant movement of the magnetic or combination of magnetic and non-magnetic assemblies can be read visually or with the aid of an appropriate instrument.

### RELATED ART

### US Patent Documents

U.S. Pat. No. 3,970,518. 1976. I. Giaever—Magnetic separation of biological particles

U.S. Pat. No. 4,935,147, 1990. E. F. Ullman, V E Ghazarossian, N Kurn—Particle separation method

U.S. Pat. No. 4,988,618, 1991. M. K. Li, J Kessler—Magnetic separation device and methods for use in

U.S. Pat. No. 5,076,950. 1991. E. F. Ullman, V E Ghazarossian, N Kurn—Magnetic composition for particle separation

U.S. Pat. No. 5,183,638. 1993. K. Wakatake—Automatic immunity analysis apparatus with magnetic particle separation

U.S. Pat. No. 5,186,827, 1993. P. A. Liberti, B P Feeley—Apparatus for magnetic separation featuring external magnetic means

U.S. Pat. No. 6,432,630, 2002. G. Blankenstein—Micro-flow system for particle separation and analysis heterogeneous assays

### BACKGROUND OF INVENTION

1. Field of Invention

This present invention relates to a rapid visual or non-visual test using non-magnetic and magnetic solid phases

2. Description of related art

Immunomagnetic separation using paramagnetic particles coated with the appropriate ligand has been used for the isolation of various components from solutions or suspensions [1]. Non magnetic colloidal, nanoparticle and microparticle surfaces have been used in in vitro diagnostics in conjunction with similar magnetic surfaces for the separation or detection of various analytes. The various approaches which employ these entities have not been used as complete devices for visual diagnostic evaluation. In particular, non-magnetic and magnetic entities have not been combined in a single embodiment for the purpose of detecting an analyte

### DEFINITIONS

1. Analyte: Any substance being analyzed
2. Nucleic acid: Group of complex compounds, composed of purines, pyrimidines, carbohydrates, and phosphoric acid. Nucleic acids in the form of DNA and RNA control cellular function and heredity
3. Colloid: A substance microscopically dispersed evenly throughout another one
4. Nanoparticle: A microscopic particle whose size is measured in nm. The term is usually reserved for particles with dimensions less than 100 nm
5. Microparticle: The term is usually used for particles ranging 300 to 700 nm.
6. The terms “nanoparticles” and “microparticles” are sometimes used interchangeably
7. Magnetic particle: A particle having magnetic properties
8. Magnetic field: An invisible field that exerts magnetic force on certain substances.
9. Conjugate: The union of soluble or insoluble entities
10. Complex: Resultant crystalline or aggregated reactants

### SUMMARY OF THE INVENTION

In vitro diagnostic techniques have been extensively reported over the years and have formed the basis for the current practice today. Some of the methods include radioimmunoassay, fluorescence techniques, agglutination, enzyme-linked-immunoassay (ELISA), lateral flow immunochromatography, electrochemistry and a variety of nucleic acid tests. The art is well established and will not be reviewed in this application. The use of paramagnetic particles for separation of biological entities is not novel. Configurations associated with the use of these particles are varied and are somewhat dependent upon the biological entity and the physical properties to be employed in the application. The gambit of these applications employ a magnetic field for separation coupled with a physical property such as fluorescence for visualization. Though applicable in a laboratory or diagnostic setting, such a configuration is not viable in a clinical or bedside application. The primary drawback to current methods lies with the requirement of specific, expensive and at times large equipment for visualization and measurement.

The invention is a multiple nanoparticle system for the visual detection of biological, chemical and other environmental agents dissolved within a fluid medium. In a preferred embodiment, the particles are composed of a non-magnetic material and a magnetic or paramagnetic material encapsulated within a nonmagnetic material. This outer material may be characterized as a non-metallic and capable of binding biological, chemical and other environmental agents. The morphology, geometry, particle dilutions surface chemistries, functionalizations, conjugated entities of the particles are to be identified and optimized and are dependent upon the particular application.

In order to remove repetition, biological, chemical and other environmental agents will be subsequently referred to as entities. The identification of entity presence is achieved by binding the sample which is thought to contain the entity to the non-magnetic particles. The entity sample may be obtained by means achieved by those skilled in the art and is subsequently deposited with a container containing the dilution of non-magnetic particles. The entity binding may be accomplished by any means practiced by those skilled in the art. Examples of such a binding process include but are not restricted to methods involving conjugation, incubation, fluid dilutions and chemical and physical reactions.

The first particle consists of a non-magnetic material that displays the property of color and possesses surface characteristics that will permit the adhesion of biological or biochemical agents to the surface. The second particle is a magnetic or paramagnetic material is contained within a non-magnetic surface capable of accepting an agent that will bind with both the surface of the second particle as well as display an affinity for the entity to be detected. Binding of this agent to the non-magnetic surface of the second particle may be accomplished through binding methods exemplified previously and performed by those skilled in the art.

Detection of the entity is achieved by combining the nonmagnetic particles with the entity bound to the surface with particles containing the magnetic (or paramagnetic) core which have the binding agent deposited on the encapsulating surface. The combination of these two particles may be performed in any suitable container and is non-specific to the invention. In addition, the mixing time and any agitation will be dependent upon the specific agents involved. Upon separation, a positive result will be observed if the specific affinity of the agent (on the magnetic particles) to the entity (on the non-magnetic particles) combines to form a stable network consisting of both magnetic and non-magnetic particles. If the non-magnetic particles contain an entity that is not consistent with the affinity of the agent deposited upon the magnetic particles a negative result will be observed upon separation.

Separation of the combined particle mixture is achieved by placing a permanent or electromagnet near one end of the container and permitting a sufficient period of time to elapse. The specific time required is dependent upon the volume of fluid involved and the size of the container utilized. The effect of the magnetic field generated by the magnet will cause particles containing magnetic material to move toward the magnetic field source. In the case where entity/agent affinity is positive, the entire magnetic and non-magnetic particle network will migrate toward the magnetic source and render a solution that is the color of the diluents used to generate the particle solutions. If the entity/agent affinity is negative, only those particles containing the magnetic or paramagnetic core will migrate toward the magnetic field source. The resulting solution will contain only the diluents from the particle solutions and the non-magnetic particles. In a preferred embodiment these non-magnetic particles will possess a color. Hence the negative solution will display a color while a positive solution will display the color of the particle diluents, in the preferred embodiment these diluents will be clear.

### BACKGROUND

### Mathematical Basis of Operation

If one defines b₁ as the fluid resistance of the vessel media, m₁ as the mass of the object moving through the media and F_{mag} as the magnetic force generated by the permanent magnet In this particular case one will deal with the negative reaction. Thus, b and m will represent the corresponding values of fluid resistance and mass as related to the migration of the paramagnetic particle in isolation. The velocity of the paramagnetic particles under a negative reaction may then be described by:

$$( \frac{d {v}_{1}}{d t} ) {m}_{1} = {F}_{mag} - {b}_{1} {v}_{1}$$

where the magnetic field strength can be considered constant over the short distance to be travelled and can be solved for the velocity v₁ using an integrating factor to yield:

$${v}_{1} = \frac{{F}_{mag}}{{b}_{1}} [ 1 - {e}^{\frac{- {b}_{1} t}{{m}_{1}}} ]$$

as the resultant velocity for the paramagnetic particles under a negative reaction condition. In the case of a positive reaction the respective fluid resistance and additional mass are represented by b₂ and m₂=m₁+m, where m is the additional mass of the immunocomplex.

In a similar manner the velocity v₂ of the paramagnetic particles under a positive reaction may be expressed as;

$${v}_{2} = \frac{{F}_{mag}}{{b}_{2}} [ 1 - {e}^{\frac{- {b}_{2} t}{{m}_{2}}} ]$$

The resistance of the fluid; b, is proportional to r, the radius of the object moving through the media in the vessel. If one examines the ratio (v₁/v₂) and replaces the fluid resistance with the respective radii one obtains the following expression for the velocity ratio:

$$\frac{{v}_{1}}{{v}_{2}} = \frac{{r}_{2}}{{r}_{1}} \frac{1 - {e}^{\frac{- {b}_{1} t}{{m}_{1}}}}{1 - {e}^{\frac{- {b}_{2} t}{{m}_{2}}}}$$

The ratio of the exponential terms

$$\frac{\frac{{b}_{1}}{{m}_{1}}}{\frac{{b}_{2}}{{m}_{2}}} = \frac{{r}_{1}}{{r}_{2}} \frac{{m}_{1} + m}{{m}_{1}}$$

in a positive reaction leads to m₁+m>m₁ with r₁ approximately equal to r₂ since the smaller colored polystyrene particles are considerably smaller than the paramagnetic particles. This leads to

$$\frac{{r}_{1}}{{r}_{2}} \frac{{m}_{1} + m}{{m}_{1}} > 1$$

### With

$$1 - {e}^{\frac{- {b}_{1} t}{{m}_{1}}} > 1 - {e}^{\frac{- {b}_{2} t}{{m}_{2}}} ( t > 0 )$$

the ratio

$$\frac{{v}_{1}}{{v}_{2}} = \frac{{r}_{2}}{{r}_{1}} \frac{1 - {e}^{\frac{- {b}_{1} t}{{m}_{1}}}}{1 - {e}^{\frac{- {b}_{2} t}{{m}_{2}}}} > 1$$

indicates that a negative reaction will lead to paramagnetic particles travelling toward the magnetic source at a greater velocity than they would if connected to additional particles within a large immunocomplex (a positive reaction).

The preceding analysis demonstrates that under a positive and negative biochemical reaction between entities coated to paramagnetic and non-paramagnetic particles, the resultant velocities will be different and are indicative of the reaction type (positive or negative). Such a property can be exploited and be implemented in design of a rapid visual assay for biologic and chemical separation.

### DESCRIPTION OF THE INVENTION

The invention employs a methodology for bio-chemical agent detection encompassing a paramagnetic particle for separation as well as a second non-magnetic tracer for visualization of diagnostic results without use of equipment. Further, increased detection levels can be obtained through the measurement of the electrical resistance of the media used for the suspension of the particles. The computations presented in this work can be adapted to a variety of biological entities and aid in the development of new and portable diagnostic configurations for clinical applications.

The current embodiment of the method employs a single paramagnetic particle element possessing a polystyrene outer shell. This coating may be functionalized or contain a surfactant, Carboxyl groups or other molecules that facilitate binding with a biological entity are generally preferred. The second tracer is a non-magnetic particle suitable for binding to a biological entity that will react to the entity bonded to the surface of the paramagnetic particles. This second tracer is selected so as to possess a color so as to provide visual evidence of the tracer in solution. More specifically, a blue polystyrene particle was selected. This idealized embodiment scenario is depicted in FIG. 1 where an antigen-antibody immunologic reaction is depicted.

The lack of a magnetic field B, will result in the poles of the paramagnetic particles to be oriented in a random configuration. (FIG. 1) Under a non-zero magnetic field B, the poles of the paramagnetic particles will align with the field lines. (FIG. 2) With this in mind, the inventors propose the bounding of a target molecule or biological/chemical agent on colored particles. Earlier, and in a separate vessel, a conjugation of the target specific probe to the paramagnetic particles was also performed. (FIG. 3) The attachment of specific elements to the respective nanoparticles may be performed by means described in the public domain and is not included as being germane to the invention. The colored particles and paramagnetic particles were subsequently mixed together.

With respect to application as a diagnostic or separation test, if the target/probe couple is acceptable, a reaction between the target (conjugated on the colored beads) and the probe (conjugated on the paramagnetic particles) will occur and result in a large complex molecule containing colored and paramagnetic particles, that is a positive reaction. (FIG. 4) If an target/probe couple is not viable then the conjugated particle assemblies will not form a large array of interconnected bonded particles; a negative reaction. (FIG. 5) A magnetic field B is generated and brought into close proximity of the vessel containing the multiple particle mixture. The magnetic field located at one end of the vessel will induce the paramagnetic particles to migrate toward the magnetic pole of the bar magnet, hence resulting in a concentration of the paramagnetic particles at one end of the vessel. A similar target/probe configuration can be applied to encompass a multitude of diagnostic/separation applications.

In the case of a positive reaction, the resulting media behind the paramagnetic migration will be clear as the entire immunocomplex is drawn toward the magnet pole due to the appropriate antigen-antibody bonding that has occurred. (FIG. 6) In a negative reaction the media behind the paramagnetic concentration will remain the color of the polystyrene particles as these are not drawn toward the magnet pole and hence reside within the fluid media within the vessel. (FIG. 7)

The preceding description of the invention can be applied to any biological or chemical combination encompassing a target/probe condition that can be isolated by incorporating two species upon the respective paramagnetic and non-paramagnetic particles. In each specific case a series of optimizations will be required that are related to the media, reaction vessel, species involved and the geometrical and chemical composition of the magnetic and non-magnetic nanoparticles.

### Use of Electrical Resistance for Enhancement of Diagnostic Detection

Assays that can be visualized without additional equipment are useful and provide a significant improvement for point of care diagnostics, employment of simple and inexpensive equipment based on measurement of common physical properties can lead to increased levels of detection and sensitivity. One such method specific to the dual tracer invention presented in this application exploits the residual electrical resistance of the fluid remaining in the vessel following paramagnetic particle migration.

Such a measurement can be useful in cases where one is visually uncertain if the vessel media contains entity coated particles that may display altered binding mechanisms other than those targeted by those entities residing on the paramagnetic particles. Consider a case where coated colored polystyrene particles remain in solution. (FIG. 4)

Let rₚ and σₚ represent the radius and cross sectional area of the polystyrene particle. In addition, let N and ΔN represent the total area and total polystyrene areas respectively. In a unit volume (L²Δx), the total number of polystyrene particles is given by (nₚL²Δx) where nₚ is the number of particles per unit volume. The total polystyrene area can now be expressed as (nₚL²Δx)σₚ. The total area is given by L². The ratio of ΔN/N can now be expressed as;

$$\frac{Δ N}{N} = \frac{Δ {xL}^{2} {n}_{p}}{{L}^{2}}$$

$$σ = {n}_{p} Δ x {σ}_{p}$$

This is termed the macroscopic collision cross section which is different from the microscopic collision cross section defined as σₚ=πrₚ²[2]

Since each ΔN diverts a particle from the original path the total number in the flow decreases N and therefore

$$\frac{Δ N}{N} = - ( {n}_{p} Δ x {σ}_{p} )$$

If one integrates over a large number of particles N;

$$\frac{d N}{N} = - {n}_{p} {σ}_{p} d x$$

resulting in N=N₀e^{-xσ}^{ₚ}ⁿ^{ₚ}This expression represents the number of particles not having a collision after travelling a distance x. The mean free path can then be designated as l; [1]

$$l = \frac{Δ N}{N} x {=}_{0}^{∞} \frac{{Nn}_{p} {σ}_{p}}{{N}_{0}} x d x {=}_{0}^{∞} \frac{{Nn}_{p} {σ}_{p}}{{N}_{0}} x {e}^{- x {σ}_{p} {n}_{p}} d x$$

$$l = \frac{1}{{n}_{p} {σ}_{p}}$$

In a time Δt, the particles will travel a distance equal to vΔt. The average number of collisions per unit time is given by:

λ=v/l=vnₚσₚ

The mean free time is given by:

$$τ = \frac{1}{λ} = \frac{1}{{vn}_{p} {σ}_{p}}$$

In classical statistical mechanics, molecules are considered indistinguishable and the average number of molecules in each of the possible energy states is relatively small. One can therefore define a partition function: [1]

$${=}_{j} {g}_{j} {e}^{\frac{- {ɛ}_{j}}{KT}}$$

where εⱼ is the energy of each level and gⱼ is the degeneracy of number of energy states in the energy level.

If it is assumed that the particles do not interact except for collisions, one can consider the particle as a particle in a box similar to a square well concept in quantum mechanics. Under such a model, the energy levels are given by:[1]

$${ɛ}_{j} = \frac{{n}_{j}^{2} {h}^{2}}{8 m} {V}^{\frac{- 2}{3}}$$

where nⱼ²=nₓ²+n_{y}²+n_{z}² is a vector in n-space from the origin to any direction and is a quantum coordinate, h is Plank's constant, m is mass and V is the volume.

Therefore in a given volume the energy depends only on nⱼ. As a result all energy states of equal energy lie on a sphere of radius nⱼ. Since there is 1 point per unit volume in n-space, the total number of possible energy states up to energy level εⱼ is equal to the volume of the sphere defined by nⱼ, with x, y and z as integers and positive in value. Thus, the true volume is actually ⅛ of the total spherical volume. The total number of energy states in all levels up to and including energy εⱼ is given by

$${G}_{j} = \frac{1}{8} \frac{4}{3} π {n}_{j}^{3} = \frac{π}{6} {n}_{j}^{3}$$

The number of states in the macro level between εⱼ and (εⱼ+Δεⱼ) (i.e. the degeneration) is given by ΔGⱼ:[1]

$$Δ {G}_{j} = \frac{π}{2} ( {n}_{j}^{2} Δ {n}_{j} )$$

and represents the number of points in a thin sphere with radius Δnⱼ and thickness Δnⱼ. From the partition function:

$$\begin{matrix} {=}_{j} {g}_{j} {e}^{\frac{- {ɛ}_{j}}{KT}} \\ = {}_{j} Δ {G}_{j} {e}^{\frac{- {ɛ}_{j}}{KT}} \\ {=}_{j} \frac{π}{2} {n}_{j}^{2} Δ {n}_{j} {e}^{\frac{- {ɛ}_{j}}{KT}} \\ = {}_{j} \frac{π}{2} {n}_{j}^{2} Δ {n}_{j} {e}^{\frac{\frac{{n}_{j}^{2} {h}^{2}}{8 m} V \frac{- 2}{3}}{KT}} \\ = {}_{0}^{∞}{n}{}_{j}^{2} {e}^{\frac{\frac{{n}_{j}^{2} {h}^{2}}{8 m} V \frac{- 2}{3}}{KT}} d {n}_{j}\end{matrix}$$

From tables for the partition function the value of this integral is given as

$$= {V ( \frac{2 π mKT}{{h}^{2}} )}^{\frac{3}{2}} \qquad [ 3 ]$$

Let η be the total number of molecules with energy up to εⱼ. The average number of molecules in a macro level is then Δηⱼ. However Δηⱼ and ΔGⱼ represent the occupation number and N̅ⱼ and the degeneracy gᵢ of a single energy level with respect to both the Maxwell-Boltzmann and classical distribution function. One can rewrite the distribution function as:

$$\frac{\overline{{N}_{j}}}{{g}_{j}} = \frac{N}{} {e}^{\frac{- {ɛ}_{j}}{KT}}$$

resulting in:

$$Δ {N}_{j} = \frac{N}{} Δ {G}_{j} {e}^{\frac{- {ɛ}_{j}}{KT}} With$$

$${ɛ}_{j} = \frac{{n}_{j}^{2} {h}^{2}}{8 m} {V}^{\frac{- 2}{3}} = \frac{1}{2} {mv}_{j}^{2}$$

one can rearrange for nⱼ giving an expression for ΔGⱼ as

$$Δ {G}_{j} = \frac{π}{2} ( {n}_{j}^{2} Δ {n}_{j} ) = ( \frac{4 π {m}^{3}}{{h}^{3}} V ) {v}^{2} Δ v$$

Substituting

$$\underline{Z} = {V ( \frac{2 π m K T}{{h}^{2}} )}^{\frac{3}{2}}$$

into the expression for ΔNⱼ yields

$$Δ {N}_{j} = \frac{4 N}{\overline{π}} {( \frac{m}{2 K T} )}^{\frac{3}{2}} {v}^{2} {e}^{\frac{- m {v}^{2}}{2 K T}} Δ v$$

The most likely velocity can be determined by setting the derivative

$$( \frac{d Δ {N}_{j}}{d v} ) = 0$$

The most probable speed results in

$${v}_{m} = \frac{\overline{2 K T}}{m}$$

If one now replaces the velocity with the most likely velocity in the expression for the mean free time;

$$τ = \frac{1}{v {n}_{p} {σ}_{p}} = \frac{1}{{n}_{p} {σ}_{p} \frac{\overline{2 K T}}{m}}$$

The mean free time is directly related to the number of polystyrene particles remaining in the vessel media.

Define the resistivity ρ and a current density J and Ohm's law in the form: V=IR with V for voltage, I represents the current and R the resistance. For a uniform current I through a conductor of length L with cross sectional area A an induced electric filed is generated and is given by E=ρJ. The current density and voltage drop can be written as:

$$J = \frac{I}{A}$$

$$V = EL = \frac{I ρ L}{A}$$

leading to an expression for the resistance as:

$$R = \frac{ρ L}{A}$$

For n electrons per unit volume moving with a velocity vₑ the current density is parallel to J. In a time dt the electrons move a distance d=vₑdt. For a cross sectional area A perpendicular to the direction of travel, the number of electrons passing through A is given by (nvₑdt)A. For an electron of charge −q the total charge crossing the area A in a time dt is given by

−q(nA)vₑdt

yielding an expression for current density as

$$J = \frac{I}{A} = - q n {v}_{e}$$

If the electric field is zero, then the velocity vₑ is random and thus v_{e average} is also zero. If the electric field is non zero, then v_{e average} is opposite to the direction of the electric field. At time zero, let t=Δt since the last collision. Since the direction of vₑ after the last collision is random, the initial velocity immediately following a collision is given by:

$$v = {v}_{0} - \frac{q E τ}{m}$$

Since vₒ is random, v_{o average} is also zero. The resulting velocity is defined as the drift velocity v_{d} and given by:

$${v}_{d} = - \frac{q E τ}{m}$$

Using the expression for the current density with the expression for drift velocity results in:

$$J = - q n {v}_{d} = \frac{E}{ρ}$$

$$\frac{E}{ρ} = - q n ( - \frac{q E τ}{m} )$$

$$\frac{1}{ρ} = \frac{n {q}^{2} τ}{m}$$

Recall that the electrical resistance was previously defined as

$$R = \frac{ρ L}{A} = \frac{m L}{{q}^{2} n A τ}$$

Replacing τ with

$$τ = \frac{1}{{n}_{p} {σ}_{p} \frac{\overline{2 K T}}{m}}$$

and σₚ=πrₚ² results in an expression for the resistance given by:

$$R = \frac{π {n}_{p} {r}_{p}^{2} \overline{2 K T}}{{q}^{2} n A}$$

For any embodiment employing uniform vessel geometry and particle diameter at a constant temperature, the quantities in the above expression for the resistance are all constant with the exception of nₚ, the number density of polystyrene in the vessel media following paramagnetic particle migration. Such a result will permit detection levels below that of visualization with the naked eye. In cases where lower detection levels are required, external devices can be incorporated into the invention configuration that will permit measurement of electrical resistance associated with the residual particles (and biochemical entity) in the vessel.

To lower the lower detection limit the inventors employ the use of electrical resistance of the residual media behind the paramagnetic particle assembly following magnetic separation. Two electrodes are inserted within the capillary tube or other configuration of the reaction vessel so as to reside within the media at a preset separation distance from each other. Neither electrode is to reside within the paramagnetic assembly following separation. (FIG. 8) The electrical resistance between the electrodes is measured via any device capable of performing such a measurement. Comparison to a negative control will yield elevated resistance values for those samples that contain residual bio-chemical material within the media following magnetic separation. Use of resistance measurements allowed for the detection limit to be reduced to less than 0.5 ng/ml of Chlamydia antigen. The detection limit for other bio-chemical entities employing this supplemental technique is again dependent upon the parameters previously cited.

Below are the specific considerations that are associated with the invention in the case of Chlamydia trachomatis detection. The unique specifications are based upon the bio-chemical entity to be identified, the media in which the reaction occurs and the geometry of the reaction vessel. In this specific preferred embodiment, the invention consists of 0.14 μm diameter (2% suspension) blue polystyrene particles and 1.0 μm diameter (10% suspension) paramagnetic particles each in separate containers. A volume of the colored polystyrene particles are mixed with Chlamydia trachomatis serovar D elementary bodies (EBs), while an approximately equal volume of the paramagnetic particles are coated with a lipopolysaccharide-specific monoclonal antibody directed to a the genus-specific epitope, 3-deoxy-alpha-D-manno-2-octulosonic acid[4]. The coating of each respective particle may be accomplished by a variety of methods as specified in the public domain. Following the introduction of the bio-chemical entities with the respective particles, an incubation period not longer than approximately 15 minutes at room temperature is permitted. Samples of the polystyrene and paramagnetic coated particles are drawn from the respective volumes. The sample volumes are on the scale of microliters and are subsequently injected into a capillary tube. The capillary tube may be of any clear material so as to permit visualization of the magnetic separation. Following a brief time period for settling and mixing at room temperature, the capillary tube is exposed to a permanent magnet at one of the ends resulting in migration of the paramagnetic particles and visualization of the results. The above embodiment yields visual detection limits of 0.9 ng/ml for the Chlamydial antigen. The detection limits for other entities may be different from this value and is dependent upon the final configuration of the multiple particle methodology.

The colored non-magnetic particles may be of any material suitable for adhesion of biochemical agents. The paramagnetic particles may be any particles containing a magnetic core surrounded by a material suitable for adhesion of bio-chemical agents that will react with agents secured to the colored non-magnetic particles. The relative size and proportion of each particle concentration is dependent upon the specific bio-chemical agent, geometry of the reaction vessel and the media of the reaction. More specifically, the geometrical configuration of the invention includes; the nanoparticle diameters specified above, the species to be detected, the incubation times and temperatures and the materials comprising the particles and reaction vessels.

### BRIEF DESCRIPTION OF DRAWINGS

FIG. 1. Magnetic and paramagnetic nanoparticles display a unique property that when no magnetic field (B) is present, the poles of the magnetic material encapsulated by the non-magnetic material are randomly oriented.

FIG. 2. When a magnetic field (B) is present and not equal to zero, the poles of the magnetic material encapsulated by the non-magnetic material magnetic become oriented with one of the magnetic poles of the magnetic field (B)

FIG. 3. LEFT. A non-magnetic nanoparticle with the target sample bound to the surface. In the preferred embodiment, the non-magnetic particle consists of a polystyrene colloidal with a biological target mounted upon the surface. RIGHT. A magnetic nanoparticle with the probe (detector for the target sample) attached to the surface. In the preferred embodiment the magnetic particle consists of a paramagnetic material core encapsulated with a non-magnetic layer.

FIG. 4. In the case of a proper target/probe affinity non-magnetic nanoparticles with the target secured to the surface will be drawn toward magnetic nanoparticles with the probe mounted to the surface. This will result in a large network or complex of magnetic and non-magnetic nanoparticles that are bound by the target/probe reaction.

FIG. 5. In the case of a improper target/probe affinity non-magnetic nanoparticles with the target secured to the surface will not be drawn toward magnetic nanoparticles with the improper probe mounted to the surface. This will result in a distribution of magnetic and non-magnetic nanoparticles that are randomly spaced as the target/probe reaction did not result in binding.

FIG. 6. Proper target/probe affinity. The large network or complex of magnetic and non-magnetic nanoparticles that are bound by the target/probe reaction will migrate toward the source of the magnetic field. In the preferred embodiment the non-magnetic nanoparticles display a color. The result of the migration of large network or complex is a clear solution behind the nanoparticle assembly that has migrated toward the magnetic source.

FIG. 7. Improper target/probe affinity. The distribution of magnetic and non-magnetic nanoparticles that are unbound due to the improper target/probe reaction will result in migration of magnetic nanoparticles toward the source of the magnetic field. In the preferred embodiment the non-magnetic nanoparticles display a color. The result of the magnetic nanoparticle migration is a colored solution behind the magnetic nanoparticle assembly that has migrated toward the magnetic source.

FIG. 8. With the aid of proper instruments, increased sensitivity can be achieved. In the case of proper target/probe affinity where target concentration is significantly reduced, test results for the presence of the target in the sample it may not be visually definitive. Use of external instruments can be used to determine if the resulting solution behind the nanoparticle assembly that has migrated toward the magnetic source contains traces of non-magnetic nanoparticles. In the preferred embodiment the external instrumentation entails the measurement of solution resistance to, or conductivity of, electricity.

FIG. 9. Preferred embodiment of a device incorporating magnetic and non-magnetic nanoparticles for separation. The device consists of: a base (1), target/probe particle introduction area, a particle mixture (2) containing magnetic and non-magnetic particles with the target and probe mounted on the respective particle surface (3), a reaction vessel (4), a magnetic shield (5) and a magnetic source (6).

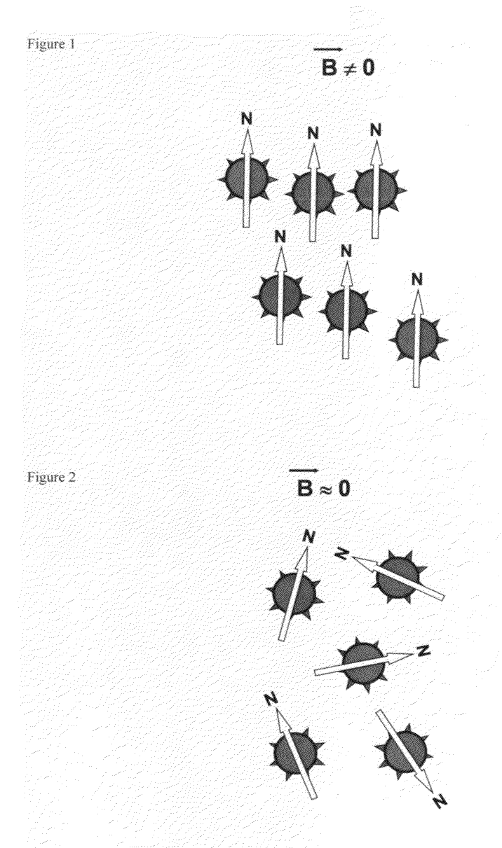

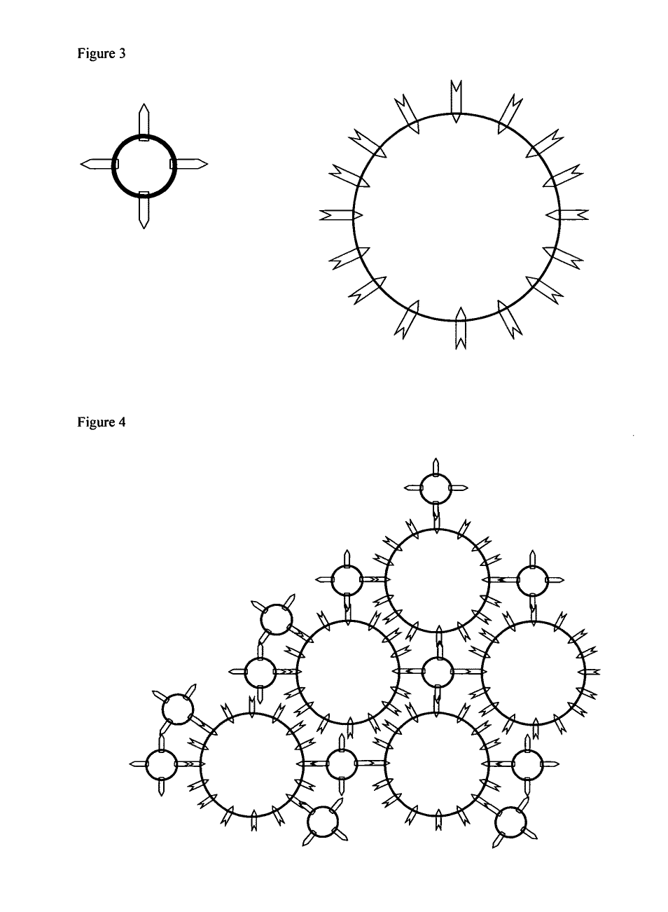

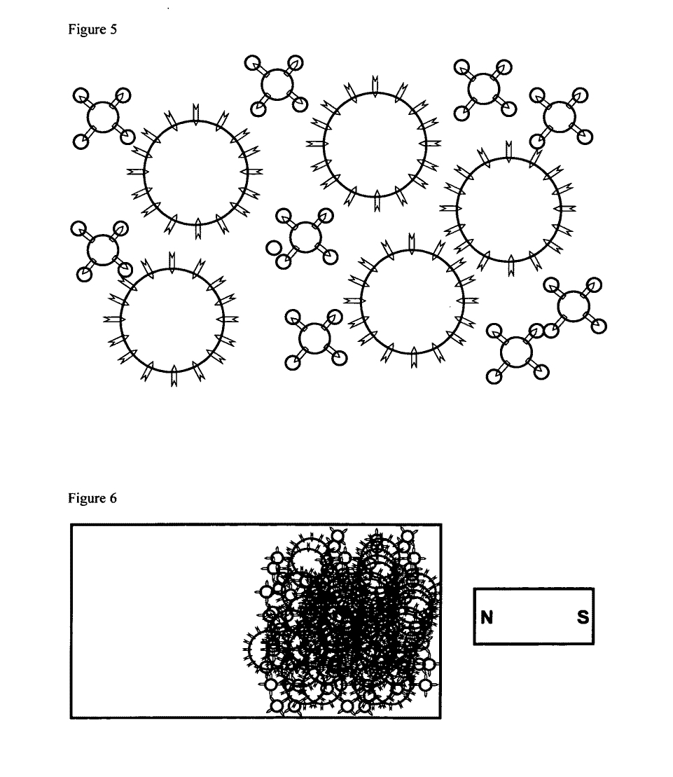

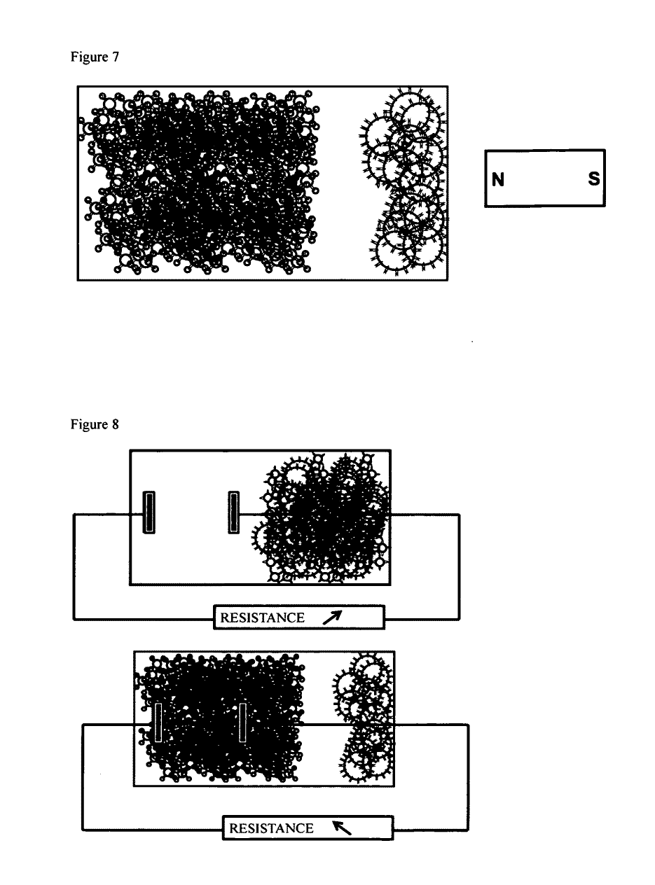

### DETAILED DESCRIPTION OF PREFERRED EMBODIMENT

Attachment of the respective target and probe to the magnetic and non-magnetic particle is achieved by those experienced in the art. In the present invention, the nanoparticles may be coated with a ligand specific for a binding partner that can bind any target molecule. Methods of coupling ligands to particles are well known in the art. For example, in one embodiment, the nanoparticles possess surface sulfate charge groups that can be modified by the introduction of functional groups such as hydroxyl, carboxyl, amine and carboxylate groups. The functional groups may be exploited for the binding of a wide variety of ligands to the nanoparticles, and are selected based on their ability to facilitate binding with the selected ligand. Conjugation of the ligands to the nanoparticle is accomplished by covalent binding or, in some cases, by adsorption of the ligand onto the surface of the nanoparticle. The techniques for adsorption or covalent binding of receptors to nanoparticles are well established in the art.

Several samples or specimens can be used with this system. These include, whole blood, plasma, serum, urine, stool, water, food extracts, dirt extracts, chemical or biochemical configurations

Samples/specimens may be contained in various diluents and buffers. Buffers may consist of a wide range of pHs. Buffers may be comprised of Carbonate, Phosphate, MES HEPES, Borate, Acetate, and others. These may or may not contain salts such as sodium chloride. Commonly used are 10 mM phosphate buffered saline (PBS), pH 7.2 and 50 mM HEPES, pH 8.0. Blocking agents may include amino acids, peptides and proteins. Other additives may include sugars, surfactants, synthetic polymers etc.

### Target/Probe Introduction

The introduction of a target or probe may be passive or active. That is, a mechanized or manual method may be used for introduction of the respective target or probe to the respective nanoparticle. For example, a pipette can be used to apply the target or probe. The sample can also be received by the capillary action or other embodiment by chromatographic, metered or mechanically assisted transfer.

### Analytes or Targets:

Analytes or targets that can be detected singly, multiple or multiplexed with this invention may include but is not limited to:
1. Microorganisms: Examples are bacteria, fungi, viruses and their components such as cell surface receptors including lipopolysaccharide (LPS) etc.
2. Genetic applications: Some examples include nucleic acids including deoxyribonucleic acid and ribonucleic acid, aptamers, oligonucleotides, polymerase chain reaction (PCR) products, reverse transcriptase per, real time quantitative PCR, cloning gene targeting, high throughput screening (HTS), capillary electrophoresis, nucleic acid (NA) sequence analysis, NA labeling and detection, gene expression analysis, single nucleotide polymorphism (SNP) analysis, recombinant DNA analysis, RNAi, etc.
3. Protein and peptide applications: Some examples include: Cardiac markers, tumor markers receptors (e.g. β-adrenergic, T and B Cell Ligands, adenosine), enzymes, interleukins, immunoglobulins of various classes, complement components, components of hemostasis, transcription factors, hormones (e.g. reproductive endocrine hormones: hCG, LH, FSH), activator and repressor proteins, phage display peptide libraries, penicillin-binding protein, cluster of designation molecules, chemotaxins, prions, lectins, etc.
4. Protein—protein interactions: Examples are transcription factors interactions, signaling molecule interactions, blood coagulation factors interactions, etc.
5. Cell signaling, Examples: MAP kinase pathway RAS pathway, NFkB pathway, NFAT pathway, etc.
6. Biomarkers, Examples: Biomolecules up/down regulate in various cardio vascular diseases, respiratory diseases, tumors and carcinomas, neurological diseases, kidney diseases, etc.
7. Drugs of abuse, such as, alcohol, amphetamines, barbiturates, benzodiazepines, cocaine, methaqualone, opioids.

Sample pretreatment may be necessary and can be uniquely applied via any means applicable by those skilled in the art. Examples include but are not limited to; heat, filtration, erythrocyte trapping, plasma defibrination, centrifugation, chromatography, emulsification, reduction, neutralization, derivatization of the target

### Particle Selection

The moieties according to this invention are nanoparticles that can be directly visualized. Any suitable insoluble microparticle, nanoparticle or colloid may be employed. For purposes of this invention nanoparticles were used. Particles consisting of polymers may include, but is not limited to polystyrenes, polyvinyl chloride, polyacrylate, nylon, substituted styrenes, polyamides, polycarbonate, polymethylacrylic acids, polyaldehydes, etc.). Other materials such as glass, agarose, polyacrylamides, polymethyl methacrylates, Sepharose, methacrylate, acrylonitrile, polybutadiene, metals, metal oxides and their derivatives (e.g. silicon, aluminium, zirconium), paramagnetic particles and colloidal gold, dextran, cellulose, and liposomes, and natural particles such as erythrocytes, pollens, fungi and bacteria. The size of the particles used in this invention is selected to optimize the binding and detection of the target, and are typically 10 to 1000 nm in diameter and in the preferred embodiment fall into this ranger. Other diameters or sizes may also work under appropriate conditions. While the particles may be of any geometrical shape, in the preferred embodiment this shape is generally spherical as this increases binding area for the respective target and probe upon the particle. In one embodiment, the nanoparticle is substantially spherical in shape. The preferred nanoparticle in the present invention is composed of dyed polystyrene.

### The Test Device: FIG. 9

The preferred embodiment of the device is seen in FIG. 9. The device may be horizontal or vertical in operation. In the preferred embodiment, the device operates on a flat surface. The base (1) may be fabricated to any desired configuration but is preferentially designed so as to rest without motion when placed on a flat surface. Further, the base (1) may be fabricated from any material so as not to interfere with the particle migration. The base (1) may be fabricated from material that can be disposable or reused following appropriate cleaning as specified in the literature. The target/probe particle introduction area (2) may be a separate component from the base or may be incorporated into the design of the base (1). The introduction area (2) may be fabricated to any desired configuration using any material but is preferentially designed so as to allow mixing of the magnetic and non-magnetic particles containing the target and probe on the respective surface. Further, the particle introduction area (2) may be fabricated from any material so as not to interfere with the particle migration. The particle introduction area (2) may be fabricated from material that can be disposable or reused following appropriate cleaning as specified in the literature. The particle mixture (3) consists of magnetic and non-magnetic particles with the respective target and probe bound to the particle surface. The assignment of the target or probe to the magnetic or non-magnetic particle is not specific and is selected based on, but not limited to, the specific target/probe couple under investigation, the geometry of the particles, solution and sensitivity and specificity required.

The reaction vessel (4) may be fabricated to any desired configuration but is preferentially designed so as to allow for transmission of the particle mixture (3) by physical means. The vessel (4) may be an open or closed design or may be excluded from the embodiment if the base (1) design incorporates such a component. These include but are not limited to mechanical, non-mechanical, electrical, chemical or other processes such as evaporation/condensation or manual transfer. In the preferred embodiment, the particle mixture (3) is transferred to the reaction vessel (4) by means of capillary flow. To isolate the particle mixture from the magnetic source so as to eliminate premature migration a magnetic shield (5) is included within the device design. The magnetic shield (5) may be fabricated from materials known for reducing the transmission of magnetic fields and can be of any geometry. In addition, the magnetic shield (5) may be deployed or retracted by any means applicable these include but are not limited to mechanical, non-mechanical, electrical, chemical or manual methods. The magnetic source (6) may be of any geometry such that it can generate a field along an axis to initiate and sustain migration of the magnetic particles within the particle mixture (3) contained within the reaction vessel (4). The magnetic source (6) may be permanent or semi-permanent (i.e., an electromagnet) or may be generated by other means by those skilled in the art. In the preferred embodiment the magnetic source (6) consists of a permanent bar magnet. The magnetic strength and positioning is such that the particle mixture (3) contained within the reaction vessel (4) can be initiated and sustained.

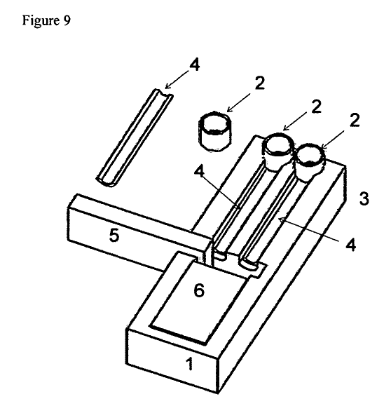

The invention presented demonstrates a platform for dual tracer particle technology that can be easily adapted to a variety of specific applications. The method is primarily visual but can be incorporated with external measurement devices to provide increased levels of detection. These devices may be based on physical properties such as, but not limited to; optical transmission, turbidity, electrical resistance or conductivity. In the preferred embodiment the measurement of fluid conductivity via resistance is utilized in order to increase the detection limits associated with the target entity (FIG. 8). The conductors may be of any geometry fabricated from electrically conductive material. In the preferred embodiment, the electrodes consist of platinum wires.

### REFERENCES

1. Olsvik, O., T Popovic, E. Skjerve, K. S. Cudjoe, E. Homes, J. Ugelstad, M. Uhlén. 1994. Magnetic Separation Techniques in Diagnostic Microbiology Clinical Microbiology Reviews. Vol. 7, No. 1. 43-54. American Society for Microbiology.
2. Sears, F. W. and G. L. Salinger, Thermodynamics, Kinetic Theory, and Statistical Thermodynamics 3^{rd} Edition (Philippines: Addison-Wesley Publishing Company, Inc., 1975).
3. Beyer, W. H. (editor) CRC Standard Math Tables 24^{th} Edition (West Palm Beach, Fla.: CRC Press Inc., 1974)
4. Fu, Y., M. Baumann, P. Kosma, L. Brade, and H. Brade. A synthetic glycoconjugate representing the genus-specific epitope of chlamydial lipopolysaccharide exhibits the same specificity as its natural counterpart, Infect Immun., 60(4), 1992, 1314-1321.

What is claimed with respect to the above invention comprises:

1. A multiple particle system for biological or biochemical separations that provides visual results indicative of the presence or absence of an analyte.

2. A multiple particle system whereby each individual particle is assigned a target or probe entity for subsequent reaction.

3. A multiple particle for biological or biochemical separations whereby the individual particles are mixed together.

4. A multiple particle system for biological or biochemical separations that may be used in a lateral (perpendicular to the force of gravity) or vertical (parallel to the force of gravity) configuration.

5. A multiple particle system for biological or biochemical separations whereby one of the particles is paramagnetic or magnetic and the other is non-magnetic.

6. A multiple particle system for biological or biochemical separations whereby one of the particles displays a color.

7. A multiple particle system for biological or biochemical separations whereby the adhesion of the biological or biochemical entities to the respective particles is performed above 0° C.

8. A multiple particle system for biological or biochemical separations whereby the adhesion of the biological or biochemical entities to the respective particles is performed above 0° C.

9. A multiple particle system for biological or biochemical separations whereby the measurement of the electrical properties of the residual media can be utilized to enhance the detection limit of the magnetic separation.

10. A multiple particle system for biological or biochemical separations whereby the procedure is conducted above 0° C.

11. A multiple particle system for biological or biochemical separations that does not require electro-mechanical drives in order to achieve the results.

12. A multiple particle system for biological or biochemical separations that does not require electrical power in order to read the results.
